(19) United States
(12) Patent Application Publication
(10) Pub. No.: US 2026/0298922 A1
(43) Pub. Date: Oct. 1, 2026

(54) ALUMINUM OXIDE SURFACES FOR DIAGNOSTIC APPLICATIONS

(71) Applicant: PAVONIS DIAGNOSTICS INC., Edmonton (CA)

(72) Inventors: Todd Patrick William MCMULLEN, Edmonton (CA); Matthew R. NICKEL, Edmonton (CA); Ana LOPEZ-CAMPISTROUS, Edmonton (CA)

(73) Assignee: PAVONIS DIAGNOSTICS INC., Edmonton, AB (CA)

(21) Appl. No.: 18/996,546

(22) PCT Filed: Jul. 27, 2023
(86) PCT No.: PCT/CA2023/051013

§ 371 Date: Jan. 17, 2025

Related U.S. Application Data
(60) Provisional application No. 63/393,731, filed on Jul. 29, 2022.

(51) Int. Cl. G01N 33/553; G01N 21/78; G01N 33/543

(52) U.S. Cl. CPC G01N 33/553; G01N 21/78; G01N 33/54393

(57) ABSTRACT

A device or method for affinity binding on an aluminum oxide surface. A biomolecule provides for immobilizing on the aluminum oxide surface. The biomolecule includes a histidine rich domain, a metal binding domain, or a metal binding moiety to immobilize the biomolecule on the aluminum oxide surface. The histidine rich domain provides at least 4 consecutive histidine residues. The metal binding domain or the metal binding moiety is selected from phosvitin, porphyrin-containing proteins and macromolecules, and nitrogen-containing macrocyclic aminopolycarboxylic acids.

### CROSS REFERENCE TO RELATED APPLICATION

This application claims priority for U.S. Patent Application No. 63/393,731 filed Jul. 29, 2022, which is incorporated by reference herein to the extent that there is no inconsistency with the present disclosure.

### STATEMENT REGARDING SEQUENCE LISTING

The sequence listing associated with this application is provided in XML format and is incorporated herein by reference. The name of the XML file containing the sequence listing is 47011WO0. The XML file is being submitted electronically via ePCT services, concurrent with the filing of this application.

### FIELD OF THE INVENTION

This invention relates to aluminum oxide surfaces for attaching biomolecules such as proteins for use in biomedical or industrial applications. The aluminum oxide surfaces are useful for applications such as diagnostic testing, affinity assays, and isolating biomolecules of interest from complex mixtures of biomolecules.

### BACKGROUND

Over the last 50 years the need for immobilized biomolecules has grown significantly. Early efforts to immobilize biomolecules were for diagnostics such as ELISA assays. In these assays antibodies were added to polymer surfaces such as microtiter dishes. These surfaces were often polystyrene. The immobilized antibodies were only 5 to 7% active as the antibodies were randomly adsorbed to the surface. It was recognized that sensitivity of ELISA assays could be increased by an order magnitude if the antibodies were oriented on the surface such that the Fab fragments were directed away from the surface and the actual binding to the surface occurred through the Fc fragment. The need for oriented immobilized biomolecules has increased with time. Microfluidic devices, gene and protein microarrays and immunodiagnostics in general all benefit from oriented binding. Further analytical techniques such as quartz microbalances, surface plasmon resonance and capillary electrophoresis all benefit from improved oriented binding. Industrial processes such as affinity chromatography and affinity separations have improved efficiency if all the proteins were ordered on the surface properly. Equally important in all of these fields is the need to prevent nonspecific protein binding.

Protein adsorption on oxides is very important in the area of bioceramics. Titania, alumina, and zirconia are some of the more common oxide ceramics used for medical applications. Due to their excellent material properties, such as corrosion resistance, wear resistance, biocompatibility and mechanical strength, these materials are often used in medicine and dentistry as biomaterials. Protein adsorption to oxides must take into account many material and environmental aspects, such as pH, microstructure, zeta-potential, and surface reactivity.

Proteins and other biomolecules such as DNA and RNA readily adsorb onto surfaces, particularly if they are charged. Attempts to improve the adsorption process have included activating the surface chemically or physically to increase the number of charged sites. When materials such as alumina are considered, which are highly catalytic, the proteins adsorbed may be denatured and thus are not stably bound and/or do not retain their bioidentity. There is ample evidence in the literature to show that this is a common phenomena, as summarized in Applicant's earlier patent application, PCT Patent Publication No. WO 2019/161491 A1. Since the adsorption process tends to be random it can also lead to inappropriate binding which may lead to steric hindrance. If molecules are oriented inappropriately or if they pack too tightly, steric hindrance occurs. If the desired proteins do not cover the surface fully, nonspecific binding can become a significant problem.

U.S. Pat. No. 5,124,172 to Burrell et al., discloses a thin film diagnostic capable of generating a colour change when contacted with a member of a binding pair such as an antibody-antigen binding pair. The diagnostic device includes multiple layers, the base layer being an anodizable metal such as tantalum capable of generating a colour when covered by a transparent layer of suitable thickness. The base layer is covered with an anodizable aluminum layer. The device is anodized and a member of the binding pair is added as a coating to the anodized substrate. When contacted with a sample containing the other member of the binding pair, a colour change occurs on binding due to a change in the refractive index and/or thickness of the transparent layers above the base metal. The diagnostic was only demonstrated for a binding pair comprising prothrombin-antiprothrombin. As mentioned above, due to the catalytic nature of an aluminum oxide surface for most proteins, the device had limited application.

Applicant's earlier patent application, PCT Patent Publication No. WO 2019/161491 A1 discloses that an aluminum oxide surface can be refunctionalized for biomedical applications with an interface molecule having a carboxy rich domain, such as the Gla domain of a Vitamin K dependent protein such as prothrombin. The interface molecule includes a polypeptide having at least one carboxy rich domain providing at least 5 free carboxyl groups within a molecular volume of 2.2-25 nm³, the free carboxyl groups being provided by amino acids containing two or more carboxyl groups, through with the interface molecule is immobilized to the aluminum oxide surface. The interface molecule is immobilized on the aluminum oxide surface, and a biomolecule or a cross linking agent is attached to the interface molecule. This aluminum oxide/interface molecule structure or device provides a base, for example, for a diagnostic device or an affinity assay, and in a manner that the biomolecule retains its biological identity, and remains attached to the interface molecule in a stable manner.

A further diagnostic device based on prothrombin as an interface molecule for an aluminum oxide surface is disclosed in PCT Patent Publication No. WO 2021/119814 A1 to SN Biomedical Inc.

### SUMMARY

One important discovery demonstrated in this disclosure is that a biomolecule including a histidine rich domain having four or more consecutive histidine residues refunctionalizes an aluminum oxide surface through the immobilization of the biomolecule on the aluminum oxide surface. A further discovery demonstrated in this disclosure is that a biomolecule including a metal binding domain or a metal binding moiety refunctionalizes an aluminum oxide surface through immobilization of the biomolecule thereon. The immobilized biomolecule on the aluminum oxide surface provides the basis for biomedical applications such as diagnostic systems and methods, kits, protein purification and detection, and affinity assays, in that the biomolecule, once immobilized via the histidine rich domain, the metal binding domain or the metal binding moiety, is shown to retain its biological identity and is stable for analysis. Having shown that these techniques result in biomolecules being stably immobilized on an aluminum oxide surface, while retaining the biological activity of the biomolecule, the inventors provided further advances by demonstrating visual diagnostic devices and methods of testing for affinity binding in which the aluminum oxide surface is provided on a reflective metal such as tantalum capable of generating a colour (a first visible interference colour) when covered by a porous layer of aluminum oxide. A colour shift or a change in the colour of the first visible interference colour is used to reliably denote binding of an analyte of interest to the immobilized biomolecule on the aluminum oxide surface.

In one broad aspect, the disclosure provides a device for affinity binding, including an aluminum oxide surface; and a biomolecule including a histidine rich domain, a metal binding domain or a metal binding moiety to immobilize the biomolecule on the aluminum oxide surface. In some embodiments, the histidine rich domain provides at least 4 consecutive histidine residues, such as a (HIS)6 tag. In some embodiments, the metal binding domain or the metal binding moiety is selected from phosvitin, porphyrin-containing proteins and macromolecules, and nitrogen-containing macrocyclic aminopolycarboxylic acids. In some embodiments, the metal binding domain or the metal binding moiety is selected from phosvitin, porphyrin-containing proteins and macromolecules having interlinked pyrrole rings, and nitrogen-containing macrocyclic aminopolycarboxylic acids having a cyclic core providing at least 10 ring members of which at least 3 are nitrogen atoms.

In some embodiments the biomolecule is immobilized on the aluminum oxide surface, while in other embodiments, the biomolecule and the aluminum oxide surface are provided as separate components for subsequent immobilizing. In some embodiments the biomolecule is immobilized on the aluminum oxide surface by physical adsorption.

In another broad aspect, the disclosure provides a method of testing whether affinity binding has occurred to an analyte of interest. The method includes:
a) providing an aluminum oxide surface;
b) providing a biomolecule specific for affinity binding to the analyte, the biomolecule including a histidine rich domain, a metal binding domain or a metal binding moiety to immobilize the biomolecule through physical adsorption on the aluminum oxide surface, wherein the histidine rich domain provides at least 4 consecutive histidine residues; and wherein the metal binding domain or the metal binding moiety is selected from phosvitin, porphyrin-containing proteins and macromolecules, and nitrogen-containing macrocyclic aminopolycarboxylic acids.
c) either i) immobilizing the biomolecule on the aluminum oxide surface and then contacting with a sample for affinity binding to the analyte, or ii) contacting the biomolecule with a sample for affinity binding to the analyte and then immobilizing the biomolecule and any affinity bound analyte on the aluminum oxide surface; and
d) detecting the presence of the analyte upon either i) binding of the analyte to the biomolecule immobilized on the aluminum oxide surface, or ii) immobilizing the biomolecule and any affinity bound analyte on the aluminum oxide surface.

In some embodiments of the method, the biomolecule is a peptide tag specific to the analyte of interest, or specific to another biomolecule which in turn is specific to the analyte of interest.

In some embodiments of the device or method, the aluminum oxide surface is provided on a reflective metal capable of generating a colour when covered by a layer of aluminum oxide; and the aluminum oxide surface is an anodized surface. In this manner, when the aluminum oxide surface is contacted with the biomolecule, a colour change is detected denoting immobilizing of the biomolecule. Alternatively, or in addition, when an analyte for affinity binding to the biomolecule contacts the biomolecule immobilized on, or subsequently immobilized with the biomolecule on, the aluminum oxide surface, a colour change is detected denoting affinity binding of the biomolecule to the analyte.

In some embodiments of the device or method, the histidine rich domain is located at one or more of a C-terminus of the biomolecule, an N-terminus of the biomolecule, or an accessible portion between the C-terminus and the N-terminus of the biomolecule. In some embodiments, the histidine rich domain includes six or more consecutive histidine residues. In some embodiments, the histidine rich domain includes a polyhistidine tag with a plurality of consecutive histidine amino acid residues at one or both of the C-terminus and the N-terminus of the biomolecule, wherein the plurality of consecutive histidine amino acid residues is 5-20, 6-10, 6-8 or 6.

In some embodiments of the device or method, the metal binding domain of the biomolecule is provided by the protein phosvitin or by porphyrin-containing proteins or macromolecules such as hemoglobin and myoglobin, cytochrome C, chlorophyll and verteprofin. In some embodiments the metal binding moiety of the biomolecule is provided by nitrogen-containing macrocyclic aminopolycarboxylic acids such as 1,4,7-triazacyclononane-1,4,7-acetic acid (NOTA), 1,4,7-triazacyclononane-1,4,7-phosphonic acid (NOTP), 1,4,7,10-tetraazacyclododecane-1,4,7,10-tetraacetic acid (DOTA), 1,4,7-trazacyclononane-1,4,7-phospinic acid (TRAP), and 6-amino-1,4-diazepine-triacetate (DATA).

In some embodiments of the method, the biomolecule and the analyte of interest are members of a binding pair selected from the group consisting of antibody-antigen, antibody-hapten, enzyme-substrate, enzyme-receptor, toxin-receptor, protein-protein, avidin-biotin, aptamer-aptamer target, and drug receptor-drug. In some embodiments of the method, immobilizing is by physical adsorption in aqueous media.

In some embodiments of the device or method, the biomolecule is provided on the aluminum oxide surface as a continuous or discontinuous coating, as delineated spots or lines, or as an array. In some embodiments of the device or method, the aluminum oxide surface is provided on a substrate is in the form of a particle, powder, thin film, slide, strip, filter, bead, magnetic bead, magnetic particle, or coating. In some embodiments, the aluminum oxide surface is provided by sputtering, evaporating, casting or extruding aluminum metal or an aluminum alloy, which is further anodized to provide a porous anodized aluminum oxide surface. In some embodiments, the aluminum oxide surface is provided by RF sputtering, reactive sputtering or chemical vapour depositing aluminum oxide onto the substrate. In some embodiments, the device is in the form of a kit.

In some embodiments, the method further includes, after detecting, contacting with a further sample to test for a secondary analyte of interest, wherein the secondary analyte of interest is capable of binding to the analyte detected in step d).

In some embodiments, the method further includes, after detecting, regenerating the aluminum oxide surface for reuse by:
i) removing the analyte and the histidine immobilized biomolecule by treating with an acid such as EDTA, H₂CO₃, or sodium bicarbonate; and
ii) optionally recharging the aluminum oxide surface with an oxidizing agent such as hydrogen peroxide.

In another broad aspect, the disclosure extends to the use of a biomolecule including a histidine rich domain, a metal binding domain or a metal binding moiety to immobilize the biomolecule on an aluminum oxide surface, wherein the histidine rich domain provides at least 4 consecutive histidine residues, and wherein the metal binding domain or the metal binding moiety is selected from phosvitin, porphyrin-containing proteins and macromolecules, and nitrogen-containing macrocyclic aminopolycarboxylic acids.

In another broad aspect, the disclosure extends to a method of testing whether affinity binding has occurred to an analyte of interest. The method includes:
a) providing an aluminum oxide surface on a reflective metal capable of generating a colour when covered by a layer of aluminum oxide, the aluminum oxide surface being a porous anodized aluminum oxide surface having a refractive index in the range of 1.2 to 1.4 to provide a first eye visible interference colour;
b) providing a biomolecule specific for affinity binding to the analyte, the biomolecule including a binding domain or binding moiety to immobilize the biomolecule on the porous anodized aluminum oxide surface;
c) either i) immobilizing the biomolecule on the porous anodized aluminum oxide surface and then contacting with a sample for affinity binding to the analyte, or ii) contacting the biomolecule with a sample for affinity binding to the analyte and then immobilizing the biomolecule and any affinity bound analyte on the porous anodized aluminum oxide surface; and
d) detecting a subsequent eye visible resulting interference colour different from the first eye visible interference colour denoting the presence of the analyte upon either i) binding of the analyte to the biomolecule immobilized on the porous anodized aluminum oxide surface, or ii) immobilizing the biomolecule and any affinity bound analyte on the porous anodized aluminum oxide surface.

In some embodiments of the method of testing, immobilizing the biomolecule on the porous anodized aluminum oxide surface produces a second eye visible interference colour different from the first eye visible interference colour denoting stable immobilizing of the biomolecule on the porous anodized aluminum oxide surface, wherein the subsequent eye visible interference colour is different from the second eye visible interference colour denoting the presence of the analyte.

In another broad aspect, the disclosure extends to a method of testing whether affinity binding has occurred to an analyte of interest. The method includes:
a) providing an aluminum oxide surface on a reflective metal capable of generating a colour when covered by a layer of aluminum oxide, the aluminum oxide surface being a porous anodized aluminum oxide surface;
b) providing a biomolecule specific for affinity binding to the analyte, the biomolecule including a binding domain or binding moiety to immobilize the biomolecule on the porous anodized aluminum oxide surface;
c) either i) immobilizing the biomolecule on the porous anodized aluminum oxide surface and then contacting with a sample for affinity binding to the analyte, or ii) contacting the biomolecule with a sample for affinity binding to the analyte and then immobilizing the biomolecule and any affinity bound analyte on the porous anodized aluminum oxide surface; and
d) detecting a subsequent eye visible interference colour different from the first eye visible interference colour denoting the presence of the analyte upon either i) binding of the analyte to the biomolecule immobilized on the porous anodized aluminum oxide surface, or ii) immobilizing the biomolecule and any affinity bound analyte on the porous anodized aluminum oxide surface;
wherein a combined thickness of the biomolecule and one or more layers of the affinity bound analyte is maintained in a range below 42 nm such that the subsequent eye visible interference colour is a first or second order interference colour.

### DETAILED DESCRIPTION

### Definitions and Terminology

Certain terms used herein and in the claims are defined and clarified hereinbelow.

“Affinity binding” refers to the selectivity of a biomolecule to a bind to another molecule such as another biomolecule. Exemplary biomolecules for affinity binding include peptide tags specific to another biomolecule, and biomolecule which are complementary members of a binding pair, for example, antibody-antigen, antibody-hapten, enzyme-substrate, enzyme-receptor, hapten-hormone, toxin-receptor, protein-protein, avidin-biotin, aptamer-aptamer target, protein-drug, and drug receptor-drug.

“Alumina” and “aluminum oxide” are used synonymously herein, and includes an oxide formed on the surface of aluminum or an aluminum metal alloy, whether the oxide is native or formed, for example by sputtering or by anodizing to provide an aluminum oxide. The aluminum oxide surface may be formed on a substrate or a support, such as on a particle, powder, bead, magnetic bead or particle, thin film, slide, strip, filter, or coating. The aluminum oxide surface may be formed by sputtering, RF sputtering, reactive sputtering, chemical vapour deposition, evaporating, casting, or extruding aluminum metal or an aluminum alloy, and may be further anodized to provide a porous anodized aluminum oxide surface, wherein “porous” indicates that the material contains a continuous network of pores throughout its volume.

“Amino acids” and “amino acid residues” described herein are typically in the “L” isomeric form, however, residues in the “D” isomeric form can be substituted for any L-amino acid residue, as long as the desired functional property of binding is retained by the polypeptide. NH₂ refers to the free amino group present at the amino terminus, or N-terminus, of a polypeptide, while COOH refers to the free carboxyl group present at the carboxyl terminus, or C-terminus, of a polypeptide. Abbreviations for amino acid residues are as follows, showing, in order, the one-letter symbol, the three-letter symbol, and the full name of the amino acid. Y, Tyr, tyrosine; G, Gly, glycine; F, Phe, phenylalanine; M, Met, methionine; A, Ala, alanine; S, Ser, serine; I, Ile, isoleucine; L, Leu, leucine; T, Thr, threonine; V, Val, valine; P, Pro, proline; K, Lys, lysine; H, His, histidine; Q, Gln, glutamine; E, Glu, glutamic acid; W, Trp, tryptophan; R, Arg, arginine; D, Asp, aspartic acid; N, Asn, asparagine; C, Cys, cysteine. Amino acid residue sequences are represented herein by formulae whose left and right orientation is in the conventional direction of amino terminus to carboxyl terminus. Furthermore, it should be noted that a dash at the beginning or end of an amino acid residue sequence indicates a peptide bond or non-standard peptide linkage to a further sequence of one or more amino acid residues.

“Analyte” refers to the specific component of a sample that is of interest for affinity binding and/or detection in a chemical or biochemical analysis.

“Antibody” includes polyclonal or monoclonal antibodies, intact antibody molecules or functional antibody fragments that are sufficient to bind to an analyte of interest, such as Fab, Fab′, F(ab)2, F(ab′)2, Fv, scFv, diabodies, linear antibodies, single-chain antibody molecules, and multispecific antibodies formed from antibody fragments.

“Aqueous media” as used herein for immobilizing on an aluminum oxide surface refers to water based solutions, which may include one or more buffers, but which excludes chemical reagents or chelating metals for covalent and/or ionic bonding.

The terms “bioidentity” and “biological identity” are used synonymously to refer to the structural and chemical property of a biomolecule that make it recognizable or specific to binding to other biomolecules, cells or tissues. For example, when an antigen is immobilized it is said to have retained its bioidentity if an antibody that was developed to it in an animal model, in vitro system, or computational model, binds to it with substantially the same specificity and sensitivity as it would if it were free in solution. As used herein, bioidentity does not necessarily require retained biological activity in a biomolecule, for example in an enzyme, it is sufficient that the biomolecule is still recognized.

The term “biomolecule” includes molecules that interact with a biological system. In general a biomolecule (or a biological molecule) is a term for molecules or ions that are present in organisms. Biomolecules include large macromolecules (or polyanions) such as proteins, carbohydrates, lipids and nucleic acids (aptamers), or derivatives or fragments thereof. Biomolecules also include small molecules such as primary metabolites, secondary metabolites, natural products and their derivatives. Biomolecules are usually endogenous, but may be exogenous. For example, pharmaceutical drugs may be natural products or semisynthetic (biopharmaceuticals) or they may be totally synthetic, and as such are included in the term biomolecules. Biomolecules also extends to synthetic proteins, polypeptides, and peptides, synthetic DNA or RNA, synthetic lipids and synthetic carbohydrates. Biomolecules may be same or different one from another in a plurality of biomolecules. A biomolecule may be linked to another biomolecule, and may provided affinity binding to a single type of molecule or to multiple molecules.

“Buffer” refers to a solution that resists changes in pH when acid or alkali is added to it. Typical buffers include a weak acid or alkali together with one of its salts.

The term “carboxy rich domain” includes polypeptides providing at least 5 free carboxyl groups within a molecular volume of 2.2-25 nm³, the free carboxyl groups being provided by amino acids containing two or more carboxyl groups, for example by one or more of the amino acids selected from the group consisting of aspartic acid (Asp), glutamic acid (Glu), and gamma-carboxyglutamic acid (Gla). Examples include Vitamin K dependent proteins, a fragment thereof containing a Gla domain, or a fragment thereof containing a modified Gla domain. More particular examples include a protein, a Gla domain of a protein, or a modified Gla domain of a protein in which one or more of the Gla residues are substituted with Glu, Asp, Glu-Glu, Glu-Asp, Asp-Glu or Asp-Asp, wherein the protein is selected from the group consisting of prothrombin, Fragment 1 of prothrombin, protein S, coagulation Factor IX, Factor X, Factor VII, protein C, matrix Gla protein, and bone Gla protein. Interface molecules with carboxy rich domain are described in carboxy rich domain in PCT Patent Publication No. WO 2019/161491 A1, the teachings of which are incorporated herein by reference.

The terms “colour change” and “colour shift” both refer to changes in one or both of the hue and intensity of reflected light that can be detected by the naked eye (i.e., “eye visible”) and/or by other appropriate colorimetric or spectrophotometric equipment used to measure changes in the wavelength or frequency of reflected light from a surface. For example, in some embodiments, differences in hue for polychromatic light sources can be detected by the naked eye for visible wavelength colour changes and/or by RGB (Red, Green, Blue) coordinates of a digital image. The RGB coordinates can be used to detect colour changes in the visible spectrum not detectable by the naked eye, and may be analyzed using mathematical and/or software assisted techniques. Models and tools for analyzing colour change/colour shift are well known, including for example, RGB coordinates, CMYK (Cyan, Magenta, Yellow and Key) and HEX (Hexadecimal) colour models. In some embodiments, differences in intensity can be measured, for example as units of LUX or lumen/m².

The terms “covalent binding”, “covalent bonding” and “covalent bonds” refer to interatomic linkages resulting from the sharing of an electron pair between two atoms, for example in the formation of covalent bonds between histidine residues, between the histidine rich domain and the biomolecule, and between biomolecules such as fusion proteins. Typically, when the biomolecule is a polypeptide such as a protein or a peptide (natural or engineered), the histidine rich domain is covalently bonded to the biomolecule through peptide bonds.

The term“domain” refers to a group of amino acids within a protein or polypeptide which is identifiable by function, properties or structure from other parts of the protein or polypeptide.

“Immobilize”, “immobilized”, and “immobilization” with respect to a biomolecule means that the biomolecule is substantially restrained or adhered on an aluminum oxide surface, for example via a histidine rich domain, resulting in reduction or loss of mobility.

“Interference colour” refers to colour produced by light reflected from different surface layers, such as a reflective base layer, for example tantalum, beneath a semi-reflective layer, for example aluminum oxide or porous anodized aluminum oxide. Differences in interference colours, observed as a “colour change” or a “colour shift”, result from differences in the optical path length, including differences in the refractive index and/or thickness, of the surface layers. It will be understood that colour change/colour shift, as used herein, includes differences in interference colours observed as colour to no colour and/or no colour to colour.

“Peptide tag” refers to any peptide sequence that specifically binds to another moiety with affinity for the tag. Peptide tags include for example purification tags and epitope tags. Exemplary peptide tags include strep-tags, flag-tags, c-myc-tags, HA-tags, epitopes and glutathione.

“Polypeptide”, “protein” and “peptide” are used interchangeably and include any peptide-linked chains of amino acids, regardless of length or post-translational modification, and as used herein includes proteins, whether natural or engineered, fragments of such proteins, fusion proteins, and amino acid sequences derived from such proteins, and fragments thereof.

“Physical adsorption” refers to immobilization of a biomolecule to an aluminum oxide surface, for example through a histidine rich domain, whereby the biomolecule is physically adsorbed or attached on the aluminum oxide surface through one or more of forces including van der Waals forces, hydrophilic-hydrophobic interactions, dipole-dipole forces, and hydrogen bonds between the aluminum oxide surface and the histidine rich domain. Physical adsorption as used herein excludes chemical bonding including covalent bonds.

“Reflective” includes semi-reflective or fully reflective, wherein “semi-reflective” means neither fully reflective nor fully transmissive, such as 30 to 70% reflective, or 40 to 60% reflective.

“Sample” includes any biological or environmental material suspected of containing one or more analytes of interest, and includes a sample which lacks the analyte such that the test for the analyte is negative. Biological samples include, for example, bodily fluids and organic materials such as foodstuffs. A bodily fluid includes, for example, whole blood, plasma, serum, sputum, cerebrospinal fluid, pleural fluid, tissue, fecal material and the like. Environmental samples include, for example, soil, sludge, water and the like. The sample can be processed, for example centrifuged, extracted, diluted, mixed with other reagents or buffers, and/or lysed if cells are present. Alternatively, the sample can be directly placed in contact with the testing surface.

### DESCRIPTION OF EXEMPLARY EMBODIMENTS

This disclosure extends to devices for affinity binding, diagnostic systems and methods for testing whether affinity binding has occurred to an analyte of interest. The device or diagnostic system includes an aluminum oxide surface, and a biomolecule to be immobilized on an aluminum oxide surface, or a biomolecule immobilized on the aluminum oxide surface. In some embodiments, the biomolecule includes a histidine rich domain providing at least 4 consecutive histidine residues for immobilizing the molecule on the aluminum oxide surface. In some embodiments, the biomolecule includes a metal binding domain or a metal binding moiety to immobilize the biomolecule to the aluminum oxide surface.

In embodiments relating to a diagnostic system, the biomolecule is specific to the analyte of interest. The method of testing includes:
a) providing an aluminum oxide surface;
b) providing a biomolecule specific for affinity binding to the analyte, the biomolecule including a histidine rich domain, a metal binding domain or a metal binding moiety to immobilize the biomolecule through physical adsorption on the aluminum oxide surface, wherein the histidine rich domain provides at least 4 consecutive histidine residues; and wherein the metal binding domain or the metal binding moiety is selected from phosvitin, porphyrin-containing proteins and macromolecules, and nitrogen-containing macrocyclic aminopolycarboxylic acids.
c) either i) immobilizing the biomolecule on the aluminum oxide surface and then contacting with a sample for affinity binding to the analyte, or ii) contacting the biomolecule with a sample for affinity binding to the analyte and then immobilizing the biomolecule and any affinity bound analyte on the aluminum oxide surface; and
d) detecting the presence of the analyte upon either I) binding of the analyte to the biomolecule immobilized on the aluminum oxide surface, or ii) immobilizing the biomolecule and any affinity bound analyte on the aluminum oxide surface.

In some embodiments, a biomolecule with a histidine rich domain, a metal binding domain or a metal binding moiety is immobilized on an aluminum oxide surface as a continuous or discontinuous coating, as delineated spots or lines, or as an array. In some embodiments the aluminum oxide surface is provided on a substrate in the form of a particle, powder, thin film, slide, strip, filter, bead, magnetic bead, magnetic particle, or coating.

Histidine rich domains are used commercially in metal affinity chromatography (IMAC) as molecular ‘anchors’ that bind through divalent cations such as nickel, copper or zinc, typically to an agarose based resin modified by nitrilotriacetic acid (NTA) bound to a solid support. IMAC has been widely used for protein purification. The process is based on chelation of the heavy metal ions and the histidine residues to the NTA matrix. The metal based chelate resin is charged with nickel or other divalent cations which are selective for the histidine rich domain. The binding of the protein to the metal cation does not occur without the histidine rich domain. In the IMAC process, the histidine rich domain is often referred to as a histidine tag. One typical tag, the (HIS)6 tag, is a polyhistidine sequence with six consecutive histidine residues present at the C- or N-terminus of a protein which is meant to be purified. Consecutive histidine residues, or histidine tags are widely known and used in molecular biology. Histidine rich domains are found in many proteins and tags, such as Zn2+ transporters, prion proteins, histidine-rich glycoproteins (HRG), some snake venoms and antimicrobial peptides.

### i) Immobilizing Biomolecules on Aluminum Oxide Surface

### a) Histidine Rich Domain Biomolecules

Through experimental efforts, the inventor determined that a biomolecule with a histidine rich domain is capable of being immobilized on an anodized aluminum oxide surface in a manner such that the biomolecule retains its biological identity, and remains attached to the aluminum oxide surface in a stable manner while retaining the functionality of the biomolecule. In some embodiments, the aluminum oxide surface is exposed to a solution of the biomolecule linked by covalent binding to a polyhistidine tag. Due to the above-noted and widely used IMAC techniques, most proteins are commercially available in a form containing a histidine rich domain, for example with a polyhistidine tag with consecutive histidine residues ranging from 4-20. Most commonly, the polyhistidine tag is a polyhistidine tag with 6 consecutive histidine residues, known as (HIS)6 or HIS6. Thus, in some embodiments, the histidine rich domain includes four or more consecutive histidine residues, such as six consecutive histidine residues. In some embodiments, the histidine rich domain includes a polyhistidine tag with a plurality of consecutive histidine amino acid residues at one or more of the N-terminus, the C-terminus, or between the N-terminus and the C-terminus of the biomolecule, wherein the plurality of consecutive histidine amino acid residues is 5-20, 6-10, 6-8 or 6. Examples 1-11 demonstrate stable immobilization of various biomolecules with polyhistidine tags.

The histidine rich domain can be genetically engineered, chemically synthesized or isolated from natural sequences. In general, the histidine rich domain provides at least 4 consecutive histidine residues as a polyhistidine tag, since a commercially available expression vector can be used so that a recombinant protein can be easily produced. In general, the polyhistidine tag sequence may be added to a protein biomolecule by incorporating the tag sequence in the primary sequence of the protein, or by grafting the tag sequence on the amino acid residue of the protein, for example. When incorporating the tag sequence in the primary sequence of the protein, it is preferable to add the tag sequence to the N-terminal or the C-terminal of the protein molecule in order to minimize the effects on the properties of the protein. However, the tag sequence may be incorporated in the internal sequence of the protein. In this case, the tagged protein may be prepared by preparing an expression vector provided with a sequence in which a gene that encodes the protein and a gene that encodes the tag sequence are fused in a state in which the open reading frames coincide, culturing E. coli or the like that is transformed by the expression vector, and separating and purifying the expressed tagged protein from the E. coli, for example. When grafting the tag sequence on the amino acid residue of the protein, the tagged protein may be prepared by coupling a tag sequence prepared by polypeptide solid-phase synthesis or the like to the purified protein, for example. The protein and the tag sequence can be coupled between the amino group and/or the imino group of the protein and the terminal carboxyl group of the tag sequences and/or between carboxyl group of the protein and the amino group and/or the imino group of the tag sequence by treating a mixture of the protein and the tag sequence using a carbodiimide reagent such as N-ethyl-N′-(dimethylaminopropyl)carbodiimide (EDC), for example.

The histidine rich domain of the biomolecule is demonstrated herein to function similarly to the carboxy rich polypeptide as described in PCT Patent Publication No. WO 2019/161491 A1, in that the histidine rich domain biomolecule refunctionalizes the aluminum oxide surface such that the biomolecule retains its biological identity and is stably immobilized on the aluminum oxide surface for applications such as diagnostic testing and affinity assays, where the biomolecule is recognized and binds to an analyte of interest in a sample.

In some embodiments, the histidine rich domain biomolecule is immobilized on the aluminum oxide surface in solution, such as aqueous media, with one or more buffers (ex. phosphate buffered saline) and additives. In some embodiments, the histidine rich domain biomolecule is applied to the anodized aluminum surface directly. In other embodiments, the histidine rich domain biomolecule is first contacted with an analyte, such as a cell lysates or a biological solution such as blood or urine, and the complexed solution is then placed in contact with the surface of aluminum oxide.

The histidine rich domain biomolecule is shown to immobilize in a reversible manner with the aluminum oxide surface, such that the surface can be recycled and reused in subsequent applications. The reversibility of immobilizing is indicated by the use of solutions with high acid or salt concentration, for example with EDTA, sodium bicarbonate or carbonic acid. The aluminum oxide surface can be recharged with an oxidizing agent such as hydrogen peroxide. Furthermore, the surface can be recycled by removing or stripping the biomolecule/analyte by destructive techniques, for example with plasma cleaning, acid or base cleaning, or thermal cleaning. The recycling and recharging steps are performed with care being taken to retain the aluminum oxide surface for subsequent immobilizing steps, as set out previously.

The histidine rich domain is located at one or more of a C-terminus of the biomolecule, at an N-terminus of the biomolecule, or between the C-terminus and the N-terminus of the biomolecule.

In some embodiments a histidine tag, as the histidine rich domain, is combined with other peptide tags such as a protein purification tags to form a bifunctional interface that then is able to immobilize to the anodized aluminum surface. In some embodiments, a protein construct is provided having a protein purification tag on one end such as Maltose binding protein (MBP), or streptavidin, or glutathione S transferase (GST), and a histidine tag on the other end of the sequence. The protein construct can be used to bind proteins with the respective peptide tag, while the histidine rich domain tag immobilizes the entire complex to the alumina oxide surface. In some embodiments, a construct with a glutathione moiety on the N-terminus and a histidine tag on the C-terminus (or the tags may be reversed), binds a GST-tagged protein with the glutathione, while the histidine tag allows the entire complex to be immobilized to the aluminum oxide surface. Other protein purification tag systems including maltose binding protein, glutathione S transferase, FLAG, and protein A/G systems, among others.

In the examples herein, engineered biomolecules with at least four consecutive histidine residues were successfully immobilized to aluminum oxide surfaces, while biomolecules engineered with less than four consecutive histidine residues, such as two or three consecutive histidine residues did not stably immobilize to the aluminum oxide surface (see Example 2). The aluminum oxide surface included a reflective base layer beneath a semi-reflective anodized aluminum oxide layer, such that an interference colour was detected on stable immobilizing of successive layers of the histidine tagged biomolecule. This initial interference colour was demonstrated to be sensitive to the initial concentration of the biomolecule, the exposure time for the biomolecule, the light intensity used for the colour detection, and refractive index of the aluminum oxide substrate. The examples also demonstrated that proteins without the histidine rich domain failed to be immobilized on the aluminum oxide surface. The examples further demonstrate a colour change compared to the initial interference colour can be produced with test samples upon successful binding of the analyte of interest to the biomolecule (ex. an antigen-antibody binding pair). Thus, the binding was quantified based on a change in interference colour resulting from a change in the optical path upon successful immobilizing of biomolecules to the aluminum oxide surface, followed by further changes in interference colour upon successful analyte binding to the immobilized biomolecules. The examples also demonstrate still further changes in interference colour upon successful binding of secondary analytes (for example secondary antibodies) to the primary analyte of interest.

In Example 1, a polyhistidine tag with six histidine residues was applied at different concentrations to the aluminum oxide surface. The isoelectric point is 8.1 and it has a net charge of positive one. The histidine tag can be genetically engineered, chemically synthesized, or isolated from natural sequences. The histidine sequences are typically at the N or C terminus of the construct, but can also be placed in an accessible segment within the central elements of the biomolecule and still retain functionality capable of immobilizing to the anodized aluminum surface.

Without being bound by the same, the immobilizing of these histidine rich constructs (biomolecule with histidine rich domain) to the aluminum oxide surface is believed to be a consequence of two features. Firstly, the histidine side chain has a high dipole moment and as such, the separation of charge is between the negative and positive elements of the histidine side chain when replicated over multiple residues, allows it to immobilize to the aluminum oxide surface via the hydroxylated as well as oxide moieties. The polyhistidine side chains thus can coordinate via electrostatic interactions with the aluminum oxide surface to allow for a stable, but reversible, immobilization to the aluminum oxide surface. Secondly, histidine can act as both hydrogen donor and acceptor and is capable of forming hydrogen bonds which, when sufficiently numbered through a string of histidine residues, or within a molecular volume, are capable of immobilizing the attached biomolecule to the aluminum oxide surface. Importantly, these physical adsorption forces are distinguished from other forms of histidine tag binding to metal affinity columns, such as in IMAC applications, where nickel is required for the histidine tag to be used because of its affinity for positively charged divalent cations. In the embodiments of this patent disclosure, divalent cations are not required or used for histidine-mediated binding to the aluminum oxide surface. In this disclosure, immobilization can be achieved in aqueous media, without other chemical reagents.

In some embodiments, the histidine rich domain can be integrated with different biomolecules, proteins, naturally occurring peptides or synthetic peptides, fusion proteins, cell lysates, or synthetically produced proteins. For example, in some embodiments, the histidine rich domain can be provided in the Fc portion of an antibody to immobilize the antibody on the aluminum oxide surface.

In some embodiments, the histidine rich domain is provided by known histidine rich proteins, or fragments thereof, with histidine repeats in the sequences, making the protein or fragments useful for immobilizing to aluminum oxide surfaces in embodiments of this disclosure. For example, proteins including CYCLIN T1, POU4F2, DYRK1A, NLK, HAND1, CBX4, YY1, FOXG1B, PRICKLE3, ZIC3, and MEC2P are representative of proteins with one or more stretches of 5 or more consecutive histidine amino acid residues that are accessible to the protein surface for immobilizing to the aluminum oxide surface. In some cases, histidine rich proteins have multiple segments of histidine repeats, including at the N or C terminus, as well as in between the N or C terminus.

b) Biomolecules with Metal Binding Domain or Metal Binding Moiety

In some embodiments, the biomolecule is immobilized on the aluminum oxide surface with a metal binding domain or a metal binding moiety. In general, the metal binding domain or the metal binding moiety is selected from phosvitin, porphyrin-containing proteins and macromolecules, and nitrogen-containing macrocyclic aminopolycarboxylic acids.

In some embodiments, such as shown in Examples 12 and 13, the metal binding domain or the metal binding moiety is selected from phosvitin; porphyrin-containing proteins and macromolecules having interlinked pyrrole rings, and nitrogen-containing macrocyclic aminopolycarboxylic acids having a cyclic core providing at least 10 ring members of which at least 3 are nitrogen atoms. Exemplary porphyrin-containing proteins and macromolecules include hemoglobin and myoglobin, cytochrome C, chlorophyll and verteprofin. The porphyrin-containing proteins and macromolecules can be used without the metal chelating ions for the pyrrole rings, for example the addition of iron ion to hemoglobin is not required for immobilizing to the aluminum oxide surface. Exemplary macrocyclic aminopolycarboxylic acids include 1,4,7-triazacyclononane-1,4,7-acetic acid (NOTA), 1,4,7-triazacyclononane-1,4,7-phosphonic acid (NOTP), 1,4,7,10-tetraazacyclododecane-1,4,7,10-tetraacetic acid (DOTA), 1,4,7-trazacyclononane-1,4,7-phospinic acid (TRAP), and 6-amino-1,4-diazepine-triacetate (DATA).

In some embodiments, for example when metal immobilizing to the aluminum oxide surface is provided by phosvitin or the metal binding domain of a protein such as a porphyrin-containing protein, the phosvitin or the protein provides an interface molecule for immobilizing to the aluminum oxide surface. In some embodiments, the metal binding protein functions as an interface molecule, similarly to that described in Applicant's earlier patent application, PCT Patent Publication No. WO 2019/161491 A1, wherein the aluminum oxide surface is refunctionalized for biomedical applications with an interface molecule having a carboxy rich domain, such as the Gla domain of a Vitamin K dependent protein such as prothrombin. Thus, for example, the phosvitin or the metal binding protein can be cross-linked to a protein of interest to serve as a diagnostic device for affinity testing, or the phosvitin or metal binding protein may be engineered as part of a larger biomolecule with functionality for affinity testing, or with a peptide tag for linking to a protein of interest.

In some embodiments, for example when metal immobilizing is with a metal binding moiety provided by a nitrogen-containing macrocyclic aminopolycarboxylic acid such as NOTA, the metal binding moiety can be synthesized or engineered as part of peptide or protein complex that can be immobilized on the aluminum oxide surface in a manner similar to the above described peptide tag immobilizing with histidine tags. In other embodiments, the macrocyclic aminopolycarboxylic acid can be engineered as an interface biomolecule such that the metal binding moiety of the biomolecule immobilizes to the aluminum oxide surface while additional functionality of the biomolecule provides a peptide tag or functionality for an analyte of interest for affinity binding.

### ii) Aluminum Oxide Surface

The aluminum oxide surface may take the form of aluminum oxides and hydroxides including calcined aluminas, the various transition aluminas (e.g. gamma, eta, delta), aluminum oxide hydroxides (e.g. boehmite), and amorphous aluminas (e.g. native oxide on aluminum, anodized aluminum and aluminum alloys).

The aluminum oxide surface may be formed on a substrate, such an optically flat substrate. Examples of substrates include a particle, powder, magnetic bead, thin film, slide or coating. The aluminum oxide surface may be formed by sputtering, RF sputtering, reactive sputtering, chemical vapour deposition, evaporating, casting, or extruding aluminum metal or an aluminum alloy, and may be further anodized to provide a porous anodized aluminum oxide surface.

iii) Biomolecules

The biomolecule is broadly defined above. For affinity binding assays, the biomolecule may be a member of a binding pair, for example: antibody-antigen, antibody-hapten, enzyme-substrate, enzyme-receptor, hapten-hormone, toxin-receptor, protein-protein, avidin-biotin, aptamer-aptamer target, protein-drug, and drug receptor-drug.

As used herein, the binding partner or bioconjugate of the biomolecule, when used in a diagnostic test for the binding pair, is referred to as an analyte of interest.

In general, affinity binding between binding pairs is referred to as molecular binding. Depending on the binding pairs, the molecular binding may be non-covalent, reversible covalent or irreversible covalent binding. Biomolecules that participate in molecular binding generally include proteins, nucleic acids, carbohydrates, lipids and small organic molecules such as drugs, whether natural, biosynthetic, synthetic or derivatized. Affinity binding may also extend to larger biounits of biomolecules, such as cell receptor molecules and viruses.

### iv) Anodization

Aluminum or anodizable aluminum alloy materials can be used for anodization to create a barrier or porous alumina layer. The main governing factors for the resulting anodic alumina films, include the electrolyte type, strength (i.e., concentration or pH), and temperature, as well as the voltage. These parameters control the porosity through pore diameter and pore wall thickness, along with the dissolution and etch rates of layers. Further treatments during oxidation can include pretreatment and post treatment. Anodizing conditions are generally well known in the art, with exemplary conditions and references relating to other anodizing conditions being set out in Applicant's earlier patent application, PCT Patent Publication No. WO 2019/161491 A1.

When alumina is soluble in the electrolyte (e.g., oxalic, sulfuric, and phosphoric acids), dissolution of Al³⁺ occurs and a porous alumina layer forms. During the initial period of anodization, a highly resistant Al₂O₃ barrier film is created on the aluminum layer. Further anodization results in the propagation of individual paths through the barrier film, which are precursors to pore formation. Next, a breakdown of the barrier film and formation of the porous structure occurs. Once porous oxide formation is complete in the aluminum layer, anodization of an underlying layer occurs (e.g. Ta). When the barrier oxide layer has completely formed current density is approximately zero.

Of particular interest for diagnostic applications are porous anodized surfaces formed on a reflective metal for use in a visual assay. The aluminum oxide surface is provided on a reflective metal, such as tantalum, capable of generating a colour when covered by a porous layer of aluminum oxide. The aluminum oxide surface is anodized, as above, to provide a porous anodized surface, such that, when contacted with a sample to test for analyte specific to the biomolecule, a colour change is detected denoting the presence of the analyte upon binding of the biomolecule and the analyte.

In some embodiments, the affinity binding device, diagnostic system or method extends to regenerating the aluminum oxide surface for reuse after conducting an initial test. The aluminum oxide surface may be regenerated by removing the analyte and the histidine immobilized biomolecule. For example, the surface may be treated with an acid or salt solution, such as EDTA, H₂CO₃, or sodium bicarbonate. This may be followed by an optional step of recharging the aluminum oxide surface with an oxidizing agent such as hydrogen peroxide.

In some embodiments, such as shown in Example 5, the anodizing of the aluminum oxide surface is performed to provide a porous anodized aluminum oxide surface having a refractive index in the range of 1.2 to 1.4. The refractive index can be varied within this range, for example by changing the anodizing conditions, such as by varying the voltage during anodizing in a range from 2V to 10V. This refractive index range is found to provide a diagnostic device having an eye visible first interference colour, which then provides changes colour shifts in the eye visible interference colours as each of the biomolecule and the analyte are added to the surface. This workable refractive index range to produce reliable eye visible colour shifts in the interference colour is found to apply for a wide range of biomolecule immobilization techniques, including immobilizing biomolecules with histidine rich domains, carboxy rich domains, and metal binding domains and metal binding moieties.

### v) Kits and Diagnostic Applications

The affinity binding device has broad application for diagnostics and for other applications. In some embodiments, such as for affinity binding applications, the biomolecule is a peptide tag specific to the analyte of interest. In such embodiments, the histidine rich domain, the metal binding domain, or the metal binding moiety of the biomolecule provides for immobilizing the biomolecule to the aluminum oxide surface, while the peptide tag binds to the analyte of interest in the sample. In other embodiments, the biomolecule is a peptide tag is specific to another biomolecule, which in turn is specific to the analyte of interest.

In some embodiments, such as for diagnostic testing, the disclosure extends to diagnostic systems or kits containing a diagnostic device providing an aluminum oxide surface on an appropriate substrate such as a slide. For visual assays or visual diagnostic devices (i.e. detecting in the visible spectrum), the diagnostic device includes an aluminum oxide surface on a reflective metal such as tantalum capable of generating a colour (interference colour) when covered by a porous layer of aluminum oxide. In such applications, the aluminum oxide surface is provided as a semi-reflective layer above the reflective layer, and is preferably provided as a porous anodized surface.

In some embodiments of visual assays or visual diagnostic testing/devices, it is beneficial to maintain a combined thickness of the biomolecule and one or more layers of the affinity bound analyte in a range below 42 nm such that the eye visible resulting interference colour is a first or second order interference colour. As demonstrated in Examples 10-11, this is found to provide reliable eye visible colour shifts in the interference colours with successive layers of binding above the porous anodized aluminum oxide surface. This maximum thickness range for biomolecules comprising proteins is found to produce reliable eye visible colour shifts in the interference colour by producing first and/or second order interference colours. This maximum thickness range is found to apply for a wide range of biomolecule immobilization techniques, including immobilizing biomolecules with histidine rich domains, carboxy rich domains, and metal binding domains and metal binding moieties.

In some embodiments of visual assays or visual diagnostic testing/devices, the use of metal compounds including an alkali metal (ex. Li, Na, K), an alkaline earth metal (ex. Mg, Ca), a transition metal (ex. Ag, Cs, Mn, Mo, Cu, Zn, Ga, Fe, Co, Ni, Cd, W), a lanthanide group element (ex. Lu) or an actinide element (ex. Th), can be used at the time of immobilization of biomolecules on the aluminum oxide surface, and/or at the time of analyte binding. Depending on the biomolecule, one or more of these metal compounds, for example nickel sulphate or calcium chloride, can assist in molecular immobilization to the aluminum oxide surface, or in the analyte binding, as demonstrated in Examples 10 and 12.

In some embodiments of visual assays or visual diagnostic testing/devices, the inclusion of nanoparticles of a reflective metal such as Au, Ag, Pt, Pd, Al, Fe, Ni, Cu, Ti, Zn or alloys of thereof, can be used to enhance the sensitivity of the assay/testing method. The nanoparticles are found to enhance the eye visible interference colours generated for biomolecule immobilization and for analyte binding, compared to binding of the biomolecule and/or the analyte without the nanoparticles. Example 14 demonstrates the use of gold nanoparticles.

In some embodiments of a visual assay or visual diagnostic device, or a kit, a histidine tagged antigen, or an antigen with the metal binding domain or the metal binding moiety, is provided on the aluminum oxide surface. Using a specific antigen, this kit detects the presence of an antibody specific to the antigen (analyte) when contacted with a test solution such as a bodily fluid, tissue or the like, since upon binding of the antigen and antibody a visible colour change occurs on the device. Detecting of secondary antibodies capable of binding to the primary antibody is also possible by contacting with successive test samples containing the secondary antibody.

In some embodiments of a visual assay or visual diagnostic device, the kit includes a diagnostic device as set out above, but conjugated with a specific antibody. This kit allows for the detection of specific antigens in a test solution, since upon binding of the antigen, a visible colour change occurs on the device. Detecting of secondary antigens capable of binding to the primary antigen is also possible by contacting with successive test samples containing the secondary antigen.

In some embodiments of a visual assay or visual diagnostic device, the kit includes a diagnostic device as set out above, but conjugated with an aptamer. The aptamer binds to its specific target molecule with a visible colour change.

In some embodiments of a visual assay or visual diagnostic device, the kit includes the aluminum oxide surface as set out above, but the histidine tagged biomolecule, or the biomolecule with a metal binding domain or a metal binding moiety, is provided separately from the aluminum oxide surface.

The diagnostic device components of the kit can be included separately, with the aluminum oxide surface provided separately from the biomolecule for immobilizing prior to the test, or the kit may include the biomolecule already immobilized on the aluminum oxide surface. The kit detects the presence of the analyte upon binding of the analyte and the biomolecule with a detectable colour change.

In addition to the diagnostic device, such kits typically include a container housing the device, and one or more other components. The kit may include pharmaceutical or diagnostic grade components in one or more containers, assay standards, testing components, buffers, regenerating solutions, oxidizing solutions etc. The kit may include instructions or labels promoting or describing the use of the device or components. Instructions may involve written instruction on or associated with packaging of the components. Instructions may also include any oral or electronic instructions provided in any manner, for example for mixing one or more components of the kit, and/or for isolating and analyzing a sample.

To facilitate quantification of distinct analytes in a sample, the assay component can employ standards for the analytes, where the standard is a predetermined amount of an analyte being detected, provided on the device to allow for the quantification of the analyte. Standards can be analyzed to produce working curves equating analyte amounts with the amount of analyte present in the sample. For visual assays, standards can include sample colours charts denoting the diagnostic device pre-testing, for a negative result, and for a positive result.

Techniques for protein printing are well known and can be used to print the histidine tagged biomolecules, or the biomolecules with a metal binding domain or with a metal binding moiety, (see for example, Delaney, Joseph T et al., McWilliam I, et al., and Li, J et al.). Protein printing, for example with an ink jet printer can be used to deposit arrays of different proteins to multiplex the tests. Each protein generates a colour that changes if their target molecules (ex. antibody, antigen, aptamer, DNA strand or RNA strand) are present. For example immunoassays can be multiplexed by placing different proteins on the aluminum oxide surface in a specific pattern. These patterns can be in the form of straight lines, curves, circles, dots, or complex patterns. They provide information on a variety of analytes in the test sample. The results are determined by changes in the colours of various portions of the printed patterns. For example, a variety of antigens can be bound to the diagnostic surface that are characteristic of specific viruses or components of the viruses, such as those that cause SARS, COVID-19, Chikungunya, Dengue Fever, Yellow Fever and Zika. The diagnostic technique identifies not only the disease, but also the serotype present, based upon colour change in specific regions. This multiplexed test determines cases in which a patient has been infected with one or more than one virus or serotype from a single sample.

In some applications, analyte-specific binding to the biomolecule can be detected using any suitable detector, and will depend on the type of test or assay being conducted. In general, the detector includes an illumination source and detection electronics. The light source can be daylight, for example for a point-of-care diagnostic, or other light sources such as LEDs, lasers and filament lamps. These sources can be used in conjunction with optical filters, polarizers, diffraction gratings and other optical components to provide a specified spectral component of light. Other forms of radiation such as bioluminescence, fluorescence, and others can be used. Excitation wavelengths my be in the visible portion of the spectrum (300-700 nm wavelength), or other wavelengths such as infrared and ultraviolet. The absorbed, reflected, or re-emitted light can then be observed and/or detected using the eye (for visible wavelengths), or using photosensitive detectors such as photodiodes or photomulipliers, in combination with spectral and/or spatial filtering.

If colour changes are below the visible detection limit and not detectable by eye, sensitive methods for detection are available. Digital image analysis, spectrophotometric and other photon counting detectors allow for the analysis of shifts in reflected wavelengths. The capture of high resolution images and digital processing is commonly used in biological studies to quantify and analyze colour patterns, so are techniques that are well understood in the art. Quantification of visible colours can be achieved with digital processing. To distinguish colours, plots can be generated using the International Commission on Illumination or Commission Internationale de l'Ecla (CIE) colour space that allows for 2D plots of chromaticity coordinates. Spectrophotometers can provide analysis with full spectrum measurements on reflectance properties, beyond what is detectable by the human eye. For very low levels of light intensity, photon counting detector assemblies (ex. photomultipliers can be used to measure the number of photons by multiplying the signal prior to detection.

One example of a suitable detector is a reflective spectrometer which measures reflectance of reflecting surfaces. Alternatively the detector can be a camera or imaging device. The detection can be at the point-of-care site, such as for a visual assay, or can be remote from the sample collection or patient site.

The methods of the present invention extend to a computer for data integration, analysis, storage and transmission in order to integrate the detected analyte-specific binding with the data acquired by any on of the data acquisition components. The binding and data thus integrated can then be analyzed and stored by the computer for subsequent access. The computer typically includes an operating system that accesses one or more algorithms and/or software to analyze data from the assay component to determine the presence and/or quantity of analytes that are being tested, for example by comparing to compiled or standard curves. The raw data and/or integrated and analyzed data are displayed on a display screen, such as a computer display or cell phone, PDA and the like.

A barcode reader can be provided for automatically entering information about an assay component. The barcode reader can be combined with a barcode activation system which identifies the test to be analyzed and automatically initiates one-point assay calibration of that particular test to reduce user errors. Each individual assay component can contain a unique barcode to be read and used to initialize the apparatus such that the appropriate algorithms are employed.

The diagnostic device can be adapted as a sample apparatus, such as a hand held device or kit, including one or more of the components such as described above.

In some embodiments, the disclosure extends to diagnostic systems or kits for use in low gravity or microgravity environments, or for use in remote settings where the diagnostic system or kit is remotely placed and read at a distance using visible light or telescopic viewing techniques. In some embodiments, the disclosure extends to use in a low oxygen or low atmospheric pressure environment such as at high elevations or in non-terrestrial environments such as low earth orbit or on a lunar surface. In some embodiments, the disclosure extends to low gravity, remote, low atmospheric pressure and other harsh environments where the anodized aluminum preparation and surfaces are used for monitoring human health, as well as for environmental assessments.

### vi) Applications

The aluminum oxide surface onto which a biomolecule with a histidine rich domain, a metal binding domain or a metal binding moiety is immobilized in accordance with this disclosure has far reaching application across many industries, including for example: protein coatings on various surfaces for a variety of products such as in biomedical applications; coatings on anodic thin films for medical diagnostic devices as visual immunoassays, coatings on anodic thin films for environmental monitoring, food indicators, indicators on clothing (smart clothes); coatings on anodic thin films for food safety diagnostic devices; coatings on anodic films in microtitre plates for ELISA testing and the like; coatings combined with other testing modalities such as lateral flow tests based on capillary action; coatings on MEMS/NEMS for diagnostics, coatings on nanoparticles for targeted therapeutic delivery; coatings on nanoparticles for contrast imaging; alumina particles for column based affinity separation applications; magnetic particles for affinity recovery or purification of biomolecules and cells; research assay to verify veracity of antibodies before testing; coatings for plastic sample cups and lids to facilitate testing, and coating on medical devices or implants.

The present invention is also illustrated by the following non-limiting examples.

### EXAMPLES

### Example 1

This example demonstrates the immobilization of a protein containing a histidine rich domain to an alumina substrate to generate changes in the visually observed interference colour when observed using white light through a polarizer at 750 from normal. Different initial interference colours were achieved, depending on the concentration of the protein. Alumina substrates were made by first sputtering tantalum to a thickness of 200 nm onto an optically flat substrate, followed by sputtering aluminum to a thickness of 110 nm on the reflective tantalum layer. The substrate was silicon wafer from University Wafer (Pittsburgh, USA, 100 mm diameter, single side polished, test grade, <100> orientation, 0-100 Ohm-cm, thickness 525+/−25 μm). For tantalum thin film, the sputtering system was KJLC (Kurt J Lesker Co.) CMS-18 Sputter System #1 (Bob), 225 nm of 3″ target, 99.95% purity tantalum, base pressure <2.0 E-6, argon flow of 10 cc, 300 W on an MDX power supply, substrate rotation speed of 3 or 4. For aluminum thin film, the sputtering system was KJLC CMS-18 Sputter System #3 (Floyd), Gun #4 (3″ aluminum), base pressure <1.0 E-7 argon gas, rotating substrate holder 20 RPM, 55 cc argon flow, 300 W. Aluminum was converted to alumina through electrochemical oxidation (anodization) which was carried out using phosphoric acid anodization at 8V with an electrolyte of 0.4 M phosphoric acid and 0.1 M oxalic acid. The anodizing details were as follows. The alumina substrate served as the anode while a cathode was prepared by wrapping a similar sized piece of sheet metal with aluminum foil. The cathode and anode were lowered into a bath. A reference electrode (Fisherbrand Accumet) was placed in the anodization bath equidistant to the cathode and anode to measure the potential across the cell. Current was measured over time and plotted using Princeton Applied Research Instruments software (PowerSuite). When the current reached zero, the process was stopped.

The biomolecule in this example was recombinant SARS CoV 2 S1 protein antigen with and without a 6 amino acid histidine sequence tag on the C-terminus (Genscript, USA). The protein, in deionized water, with and without the (HIS)6 tag, was placed on the alumina surface, 18.5 μL with increasing concentrations from 0.25 mcg/mL to 1 mg/ml, for 10 minutes in a 100% relative humidity environment and then rinsed thoroughly with deionized water. The visually observed colour shift was caused by the increase in optical path length resulting from the immobilization of the protein, as set out in Table 1. This example also reports RGB coordinates (R, G, B) from digital image analysis for the initially observed colour and for the observed colour changes at the different concentrations of the protein. All colour measurements were made using a Logitech BRIO™ 4K web camera, with the incident light from an LED source of 4350 LUX, where the pictures were taken through a polarizing filter at 750 from normal. The RGB coordinates were calculated using OpenCV in python (https://opencv.orq/). To detect the RGB values for every pixel using a digital image of at least 1280×720 pixels, 96 dpi. The recombinant SARS CoV 2 S1 protein lacking the histidine tag did not immobilize to the aluminum oxide surface and thus did not demonstrate a change in colour at any concentration.

TABLE 1

| | Colour | | Colour | RGB |
|---|---|---|---|---|
| Protein | with | RGB with | without | without |
| Conc. | HIS tag | HIS Tag | HIS Tag | HIS Tag |
| Blank slide | Yellow | 252, 176, 18 | Yellow | 252, 176, 18 |
| Water | Yellow | 251, 168, 14 | Yellow | 252, 176, 18 |
| 0.25 mcg/ml | Yellow | 251, 174, 23 | Yellow | 251, 174, 23 |
| 1.0 mcg/ml | Yellow | 252, 175, 20 | Yellow | 250, 173, 20 |
| 10 mcg/ml | Orange | 252, 173, 36 | Yellow | 253, 175, 15 |
| 50 mcg/ml | Dark orange | 250, 160, 74 | Yellow | 248, 173, 23 |
| 100 mcg/ml | Tan | 249, 146, 124 | Yellow | 251, 171, 28 |
| 500 mcg/ml | Light pink | 247, 125, 163 | Yellow | 251, 170, 22 |
| 1000 mcg/ml | Pink | 243, 93, 161 | Yellow | 252, 170, 25 |

### Example 2

This example demonstrates the immobilization of synthetic peptides containing both a histidine sequence on one terminus, and a peptide tag on the other terminus. The different peptides included histidine sequences with increasing number of consecutive histidine residues. The peptides were tested on alumina substrates for changes in a visually observed interference color when observed using white light through a polarizer at 750 from normal.

Alumina substrates were made as set out in Example 1. Aluminum was converted to alumina through electrochemical oxidation (anodization) as set out in Example 1.

The synthetic peptides included sequentially increasing lengths of histidine tags at the C-terminus, and each of the peptides on the N-terminus had a sequence capable of binding an antibody to a FLAG tag. The peptide sequences included:

SEQ ID NO 1
DYKDDDDK(LA)6HH
SEQ ID NO 2
DYKDDDDK(LA)6HHH
SEQ ID NO 3
DYKDDDDK(LA)6HHHH
SEQ ID NO 4
DYKDDDDK(LA)6HHHHH
SEQ ID NO 5
DYKDDDDK(LA)6HHHHHH.

The peptides, 250 mcg/ml in phosphate buffered saline (PBS) at pH 7.4, were placed on the alumina surface to a volume of 18.5 μL for 10 minutes in a 100% relative humidity environment and then rinsed thoroughly with deionized water. The shift in colour visibly observed was caused by the increase in optical path length which resulted from the immobilization of the peptides on the surface via a histidine rich domain in the peptides. The RGB coordinates were also reported. In each case, the thin film surface was rinsed in deionized water for 30 minutes after addition of the peptide solution. As shown in Table 2, it is apparent that immobilization of the synthetic peptide on the aluminum oxide surface did not occur until a histidine rich domain with at least 4 consecutive histidine residues was included in the biomolecule. With only two or three histidine residues, immobilization via either of the peptide tags (i.e., (HIS)2, (HIS)3 or FLAG) did not occur or immobilization was not stable, as is evident from a lack of a change in the eye visible interference colours from the initial yellow colour. With a histidine tag having only four consecutive histidine residues, some immobilization was achieved, as evidenced by a small change in colour compared to immobilization and colour change with five or six consecutive histidine residues. With a histidine tag having more than four consecutive histidine residues, as shown with the (HIS)6 tag, significant stable immobilization occurred, as is evident from the colour shift. This demonstrates that a histidine rich domain of four or more consecutive histidine residues is capable of stably immobilizing a biomolecule to the alumina substrate, and generating a change in the interference colour, while less than four histidine residues was not capable of stably immobilizing a biomolecule to the alumina substrate.

TABLE 2

| Length of C- | | RGB |
|---|---|---|
| terminus HIS Tag | Observed Colour Change | Coordinates |
| Blank Slide | Yellow | 248, 209, 65 |
| PBS Buffer | Yellow | 251, 200, 70 |
| Two histidines | Yellow | 253, 202, 66 |
| Three histidines | Yellow | 248, 199, 72 |
| Four histidines | Light Orange or Yellow-Tan | 238, 183, 85 |
| Five histidines | Tan | 223, 163, 109 |
| Six histidines | Tan | 218, 149, 112 |

### Example 3

This example demonstrates the time-dependent immobilization of a protein containing a histidine-rich domain to an alumina substrate to generate changes in the visually observed interference colours using white light through a polarizer at 750 from normal. Alumina substrates were prepared and anodized as in Example 1.

The protein, recombinant SARS CoV 2 S1 with a 6 amino acid histidine sequence tag on the C-terminus (Genscript, USA) was placed on the alumina surface, 18.5 μL of 100 mcg/ml in deionized water, in increments between 5 minutes to 120 minutes in a 100% relative humidity environment and then rinsed thoroughly with deionized water. The visually observed colour shift was caused by the increase in optical path length resulting from the immobilization of the protein, as outlined in Table 3. This example also reports the RGB coordinates from digital image analysis for the observed colour and for colour changes observed at the different times.

TABLE 3

| Protein Surface Contact | | RGB Coordinates |
|---|---|---|
| Time | Observed Colour | (R, G, B) |
| Blank slide | Yellow | 252, 213, 95 |
| Water | Yellow | 252, 219, 102 |
| 1 minute | Yellow | 252, 161, 96 |
| 5 minutes | Orange | 252, 141, 100 |
| 10 minutes | Tan | 250, 139, 122 |
| 30 minutes | Pink | 245, 130, 149 |
| 45 minutes | Pink | 243, 124, 159 |
| 60 minutes | Pink | 243, 119, 164 |
| 120 minutes | Dark pink | 238, 96, 178 |

### Example 4

This example demonstrates the immobilization of a protein containing a histidine-rich sequence to an alumina substrate to generate changes in the visually observed interference color when observed using white light through a polarizer at 750 from normal with varying light intensity. Alumina substrates were made and anodized as set forth in Example 1.

The proteins (as identified in Example 1, with a C-terminus (HIS)6 tag) were placed on the alumina surface, 18.5 μL, at 100 mcg/ml in deionized water for 30 minutes in a 100% relative humidity environment and then rinsed thoroughly with deionized water. The visually observed colour shift was caused by the increase in optical path length which resulted from the immobilization of the protein, was quantified by variations in light intensity, defined as LUX, as outlined in Table 4. This example also reports RGB coordinates from digital image analysis for the colours. This example demonstrates that a standardized light intensity measurement technique is important for reliably indicating colour change.

TABLE 4

| Light intensity | Spot | | RGB |
|---|---|---|---|
| (Lux) | Composition | Colour | coordinates |
| 250 | Blank | Maroon | 101, 52, 47 |
| 250 | Water | Maroon | 95, 46, 47 |
| 250 | Protein | Blue | 45, 30, 92 |
| 420 | Blank | Maroon | 94, 61, 73 |
| 420 | Water | Maroon | 88, 57, 73 |
| 420 | Protein | Blue | 52, 51, 118 |
| 820 | Blank | Maroon | 91, 33, 3 |
| 820 | Water | Maroon | 85, 31, 6 |
| 820 | Protein | Dark blue | 27, 10, 44 |
| 1690 | Blank | Dark orange | 102, 37, 3 |
| 1690 | Water | Dark orange | 95, 34, 4 |
| 1690 | Protein | Dark blue | 36, 13, 46 |
| 4230 | Blank | Orange | 172, 75, 3 |
| 4230 | Water | Tan | 168, 56, 37 |
| 4230 | Protein | Purple | 98, 41, 104 |

### Example 5

This example demonstrates a working range of the refractive index of the porous anodized aluminum oxide surface in order to provide visible interference colours for affinity binding diagnostic testing. A protein containing a histidine-rich sequence was immobilized to alumina substrates of varying refractive indices. The changes in the visually observed interference color were observed using white light through a polarizer at 750 from normal. Alumina substrates were made by first sputtering tantalum to a thickness of 200 nm onto an amorphous support, followed by sputtering aluminum at 110 nm on the reflective tantalum layer. Anodization proceeded as set forth in Example 1, with the exception that the changes in voltage (2V, 4V, 6V, 8V and 10V) during anodization resulted in changes in the refractive index of the porous anodized aluminum oxide surface, measured at 555 nm, of 1.21, 1.3, 1.34, 1.39, and 1.42 respectively.

The protein, 18.5 μL of 100 mcg/ml SARS CoV 2 S1 with a (HIS)6 tag at the C-terminus, in deionized water, was placed on the respective alumina surfaces for 30 minutes in a 100% relative humidity environment and then rinsed thoroughly with deionized water. The visually observed colour shift (i.e., eye visible change in interference colour) was caused by the increase in optical path length which resulted from the immobilization of the protein, as outlined in Table 5. When the diagnostic device (anodized alumina surface with immobilized protein) was contacted with an antibody specific to the protein, a further visible colour shift (i.e., eye visible change in interference colour) was observed, denoting binding of the antibody.

Similar results were obtained for diagnostic devices when proteins were immobilized on the anodized aluminum oxide surfaces with prothrombin as an interface molecule (i.e., immobilization via carboxy rich domain), that is the anodized alumina substrate provided an eye visible initial interference colour, followed by a colour shift with the prothrombin immobilized protein, and followed by a further colour shift when contacted with an analyte specific to the prothrombin immobilized protein.

The example demonstrates that successive eye visible colour shifts result for on the anodized aluminum oxide surface provided the refractive index of the alumina surface is maintained in the refractive index range between about 1.2 and 1.4. Attempts to generate eye visible interference colour changes on alumina surfaces having a refractive index outside the range of about 1.2 and 1.4 demonstrated eye visible colour shifts that were difficult to visualize or which had poor reproducibility.

TABLE 5

| Refractive Index | Spot Composition | Colour | RGB Coordinates |
|---|---|---|---|
| 1.21 | Blank | Yellow | 225, 184, 30 |
| 1.21 | Water | Yellow | 225, 185, 31 |
| 1.21 | Protein | Orange | 218, 157, 3 |
| 1.3 | Blank | Orange | 230, 134, 4 |
| 1.3 | Water | Orange | 229, 137, 7 |
| 1.3 | Protein | Pink | 223, 91, 115 |
| 1.34 | Blank | Violet | 214, 14, 157 |
| 1.34 | Water | Violet | 216, 18, 148 |
| 1.34 | Protein | Purple | 137, 18, 214 |
| 1.39 | Blank | Purple | 113, 11, 224 |
| 1.39 | Water | Purple | 130, 17, 218 |
| 1.39 | Protein | Blue | 50, 41, 221 |
| 1.42 | Blank | Blue | 36, 48, 224 |
| 1.42 | Water | Blue | 31, 64, 224 |
| 1.42 | Protein | Cyan | 16, 125, 224 |

### Example 6

This example demonstrates that proteins lacking a histidine-rich domain do not bind to the anodic alumina surface. The visually observed interference colors were observed using white light through a polarizer at 750 from normal. Alumina substrates were prepared and anodized as set forth in Example 1.

Different proteins lacking an accessible histidine rich domain (i.e., four consecutive histidine residues), including PDGFRB antibody (Santa Cruz Biotechnology, USA), VEGF antibody (Santa Cruz Biotechnology, USA), FoxE1 antibody (Santa Cruz Biotechnology, USA), ACE2 protein (In house construct), SARS CoV 2 51 protein (Genscript, USA), and 0024 antibody (Santa Cruz Biotechnology, USA), were added to the surface at 100 mcg/ml in phosphate buffered saline, pH 7.4 for 30 minutes in a 100% relative humidity environment and then rinsed thoroughly with deionized water. The lack of colour shift resulted from the lack of a significant change in optical path length due to the fact that, without a histidine rich domain, minimal protein was immobilized on the thin film surface, as outlined in Table 6

TABLE 6

| Protein composition | Colour | RGB Coordinates |
|---|---|---|
| Blank slide | Yellow | 244, 197, 70 |
| Buffer only | Yellow | 241, 197, 73 |
| PDGFRB antibody | Yellow | 244, 191, 71 |
| VEGF antibody | Yellow | 244, 194, 69 |
| FoxE1 antibody | Yellow | 244, 195, 81 |
| ACE2 protein | Yellow | 242, 187, 60 |
| SARS CoV 2 S1 protein | Yellow | 242, 177, 76 |
| CD24 antibody | Yellow | 239, 188, 73 |

### Example 7

This example demonstrates the specific antibody binding to proteins with a histidine-rich domain that are immobilized on an alumina substrate to generate changes in the visually observed interference color when observed using white light through a polarizer at 750 from normal. Alumina substrates were prepared and anodized as in Example 1.

The protein, recombinant SARS CoV 2 S1 with a (HIS)6 tag at the C-terminus (Genscript, USA), was placed on the alumina surface, 18.5 μL, 100 mcg/ml in deionized water, for 10 minutes in a 100% relative humidity environment and then rinsed thoroughly with deionized water. The antibodies, as identified below and in Table 7, at 100 mcg/ml were then added under the same conditions followed by rinsing. The colour shift was caused by the increase in optical path length resulting from the immobilization of the protein. The colour shift indicated that the protein immobilized by the histidine rich domain on the alumina surface was recognized by the SARS CoV 2 antibody, which bound and changed the overall thickness of the protein layer. The addition of antibody in this example represented an opportunity to quantify antibody levels in the applied substrate. Furthermore, for diagnostic device demonstration, the addition of the specific secondary IgG antibody (Goat anti-human IgG, Bio-Rad USA) was made. The secondary antibody generated a subsequent change in colour, demonstrating a quantifying tool for diagnostic applications. Additions of secondary antibody in the absence of primary antibody did not alter the observed colour, demonstrating a specificity of the antigen-antibody interactions on the alumina surface.

TABLE 7

| | | RGB |
|---|---|---|
| Protein Composition | Colour | Coordinates |
| Blank slide | Yellow | 243, 202, 24 |
| Water | Yellow | 244, 204, 33 |
| SARS CoV 2 S1 protein (S1) | Tan | 243, 158, 78 |
| S1 + antibody | Pink | 246, 138, 146 |
| S1 + secondary antibody (non-specific) | Tan | 243, 151, 87 |
| S1 + antibody + specific secondary antibody | Purple | 229, 66, 233 |

### Example 8

This example demonstrates immobilization of histidine-rich proteins to an alumina surface, followed by testing with varying biological samples for analyte specific binding. Alumina substrates were prepared and anodized as set out in Example 1. In this example, under the same conditions of Example 1, the SARS CoV 2 S1 protein with a (HIS)6 tag on the C-terminus (Genscript, USA), hydrated in deionized water, was added to the alumina surface, 18.5 μL, of 100 mcg/ml for 20 minutes and rinsed off with deionized water. The 1.85 mcg of the S1 protein was also added to serum, whole blood, urine, and oral secretions at the same concentration and then applied to the anodized aluminum surface. The protein immobilization to the surface induced a colour change of the slide as seen by the visually observed interference color when observed using white light through a polarizer at 750 from normal. Antibody, SARS CoV 2 S1 monoclonal antibody (Genscript, USA), binding to the S1 protein was monitored in the different test solutions, and the colour changes are shown below.

TABLE 8

| Protein and Solution Composition | Colour | RGB Coordinates |
|---|---|---|
| Blank Slide | Yellow | 250, 201, 38 |
| Water | Yellow | 247, 199, 41 |
| SARS CoV 2 S1 Protein (S1) | Tan | 240, 141, 119 |
| S1 Protein in serum | Orange | 241, 153, 109 |
| S1 Protein in serum + antibody | Dark pink | 192, 122, 163 |
| S1 Protein in whole blood | Orange | 232, 153, 118 |
| S1 Protein in whole blood + antibody | Dark pink | 199, 100, 173 |
| S1 Protein in urine | Orange | 239, 150, 122 |
| S1 Protein in urine + antibody | Dark pink | 211, 111, 155 |
| S1 Protein in oral secretions | Orange | 241, 167, 116 |
| S1 Protein in oral secretions + antibody | Dark pink | 217, 94, 190 |

### Example 9

This example demonstrates that addition of ethylenediaminetetraacetic acid (EDTA) or carbonic acid to the thin film, following physical absorption of a protein with a histidine domain on the surface, induced a change in the color of the slide that was consistent with protein being removed from the surface as seen in the changes in the visually observed interference color when observed using white light through a polarizer at 750 from normal. Alumina substrates were prepared as set out in Example 1. The protein, SARS CoV 2 S1 with a (HIS)6 tag at the C-terminus (Genscript, USA), was placed on the alumina surface, 18.5 μL of 100 mcg/mL solution in deionized water, for 10 minutes in a 100% relative humidity environment and then rinsed with deionized water. A shift in colour was observed with the addition of the protein and a second colour shift with the addition of the SARS CoV 2 S1 monoclonal antibody (Genscript, USA) as shown by the change in the RGB coordinates. When 50 mM EDTA or H₂CO₃ at pH 7.4, was added to the protein spot for two hours and then washed off with deionized water, the colour shift previously observed with absorption of the proteins or protein+antibody to the surface was abated and the colour reverted back to yellow. The shift in colours were caused by the increase, or decrease, in the optical path length due to the immobilization/physical adsorption, or freeing from the surface, of the protein. This process was reversible. Using the same slides treated with EDTA or H₂CO₃ then rinsed with deionized water, the addition SARS CoV 2 S1 with (HIS)6 tag induced a color change consistent with protein immobilization on the surface and the protein is recognized by its corresponding monoclonal antibody to generate the concomitant colour change. The results are summarized in Table 9.

TABLE 9

| Protein Composition | Colour | RGB Coordinates |
|---|---|---|
| Blank slide | Yellow | 254, 223, 108 |
| Water | Yellow | 252, 220, 100 |
| 100 mcg/ml | Khaki | 241, 163, 123 |
| 100 mcg/ml + antibody | Magenta | 192, 97, 223 |
| 100 mcg/ml + EDTA | Yellow | 240, 144, 114 |
| 100 mcg/ml + antibody + EDTA | Yellow | 237, 199, 73 |
| 100 mcg/ml + H₂CO₃ | Yellow | 231, 181, 70 |
| 100 mcg/ml + antibody + H₂CO₃ | Yellow | 244, 227, 65 |
| Reused slide + 100 mcg/ml | Khaki | 241, 151, 130 |
| Reused slide + 100 mcg/ml + antibody | Magenta | 185, 102, 180 |

### Example 10

This example demonstrates that affinity binding with diagnostic devices of this disclosure is improved when the combined thickness of the biomolecule with layers of analytes is maintained below about 40 nm, so as to generate first and second order interference colours (i.e., with eye visible changes in interference colours). The example provides visible interference colour results for varying distance off the anodized aluminum oxide surface with stacked proteins. The anodized aluminum oxide slides were prepared as outlined in Example 1. Sequential immunoglobulin proteins of varying isotypes were added to the protein coated diagnostic device to generate colour changes in the first and second order interference colours visually observed using white light through a polarizer at 750 from normal. Platelet derived growth factor receptor-α (PDGFRα) with a histidine tag ((HIS)6) was immobilized on the alumina surface under the following conditions: 18.5 μL, 100 μg/ml in deionized water and 0.05M NiSO₄, 30 minutes, in a 100% relative humidity environment. The alumina surface was then washed with phosphate buffered saline, pH 7.4, for 15 minutes, followed by a rinse with deionized water and air dried. This was followed by platelet derived growth factor AA (PDGF-AA), known to bind specifically to PDGRFα, 100 μg/ml in deionized water, 30 minutes. Then an antibody to PDGF-AA was added followed by sequential immunoglobulin protein layers added one at a time, 100 μg/ml in deionized water, 30 minutes. The first and second order interference colours were documented by the RGB coordinates with the addition of immunoglobulins that are each different and designed to recognize the different F_{c} component of the previous antibody (Table 10). In the same samples, ellipsometry was used to determine the optical path length changes to experimentally define what was predicted based on the change in thickness in the protein layer on the aluminum oxide, based on the volume of immunoglobulin proteins. S-plane polarized light was measured at 750 from normal using a J.A. Woolam M-2000 Ellipsometer from 380 to 750 nm. The following equation can be used to represent the destructive interference of light beams reflected from the front and back surfaces of a thin film:

$$O P L = m λ = 2 nd cos θ + \frac{λ}{2} ,$$

where OPL is optical path length, m is the order of interference, A is the wavelength, n is the refractive index of the thin film, d is the thickness of the thin film, and θ is the angle of incidence. Using the wavelength at the minimum intensity of each layer, the OPL can be solved for and therefore the difference in optical path length between each consecutive layer. The following equation can then be used to estimate the thickness of the protein layer from the alumina surface, assuming a uniform, flat surface:

$$Δ O P L = 2 {n}_{protein} {d}_{protein} cos θ ,$$

where nₚᵣₒₜₑᵢₙ is the refractive index of the protein layer, and dₚᵣₒₜₑᵢₙ is the thickness of this composite layer.

The estimated thickness results from Table 10 (dₚᵣₒₜₑᵢₙ) assume a uniform, densely packed layer of protein, with nₚᵣₒₜₑᵢₙ=1.5, an angle of incidence of 750 from normal, and a second order interference (m=2). The first order and second order visible color changes observed were abrogated beyond ΔOPL>110 nm or dₚᵣₒₜₑᵢₙ>42 nm.

TABLE 10

| Layer | Observed Colour | RGB Coordinates |
|---|---|---|
| Alumina surface | Yellow | (247, 132, 125) |
| PDGFRA | Purple | (227, 53, 200) |
| +PDGF-AA | Blue | (114, 106, 255) |
| +Mouse anti-PDFG-AA | Light Blue | (80, 170, 255) |
| +Goat anti-mouse | Blue-Teal | (96, 197, 255) |
| +Donkey anti-goat | Teal | (107, 204, 255) |
| +Rabbit anti-donkey | Teal | (154, 220, 252) |

### Example 11

Slides were prepared as in Example 1, but with prothrombin as an interface molecule (i.e., biomolecule with carboxy rich domain, as in PCT Patent Publication No. WO 2019/161491 A1). Sequential immunoglobulin proteins of varying antibody isotypes were added to the prothrombin interface to generate changes in the visually observed interference colours using white light through a polarizer at 75° from normal. The prothrombin (250 mcg/ml) was placed on the alumina surface, 30 minutes, 18.5 μL of deionized water in a 100% relative humidity environment and then rinsed thoroughly with deionized water. This provided a saturated surface with maximal prothrombin immobilizing. The first and second order visible interference colours were measured as documented by the RGB coordinates with the addition of immunoglobulins that are each different and design to recognize the Fc component of the previous antibody. The colours were measured as documented by the RGB coordinates and the reflectance spectrums recorded using ellipsometry as before using the same process as Example 10 to calculate the protein layer thickness. The results are provided in Table 11. Again, it was found that the observed interference color changes did not change significantly after 4 layers of protein,

$$Δ O P L > 110 nm or {d}_{protien} > 42 nm .$$

TABLE 11

| Protein Layers (IgG) | Observed Colour | RGB Coordinates |
|---|---|---|
| Prothrombin | Pink | (228, 71, 167) |
| +Sheep anti-prothrombin | Purple | (159, 38, 238) |
| +Rabbit anti-sheep | Blue | (73.5, 83.6, 243.1) |
| +Goat anti-rabbit | Blue-Teal | (71, 196, 241) |
| +Donkey anti-goat | Teal | (85, 191, 239) |
| +Rabbit anti-donkey | Teal | (134, 209, 238) |

### Example 12

This example demonstrates that metal binding domains or metal binding moieties such as proteins or peptides containing macrocyclic, heterocyclic ring systems that bind metal ions can be immobilized to an alumina substrate to generate changes in the visually observed interference color, documented by R, G, B coordinates, when observed using white light through a polarizer at 750 from normal. Alumina substrates were made and anodized as set forth in Example 1. Proteins and a peptide linked to a chelating chemical moiety (NOTA) for radioisotopes were added to the prothrombin interface to generate changes in the visually observed interference colours using white light through a polarizer at 750 from normal. The proteins were placed on the alumina surface, 18.5 μL of 100 mcg/ml in deionized water, in a 100% relative humidity environment and then rinsed thoroughly with deionized water. Antibodies when bound to the proteins on the immobilize surface were done so also under identical conditions at 100 mcg/ml for 30 minutes and the slide then rinsed with deionized water, also for 30 minutes. In the cases of hemoglobin, myoglobin metal ions were not required as part of the process of immobilization to the alumina surface. In the case of the NOTA peptide, calcium chloride was used in solution (100 mM) whereas other cations, nickel or magnesium, did not aid immobilization.

TABLE 12

| Protein | Observed | |
|---|---|---|
| Surface | Colour | RGB Coordinates |
| Blank slide | Yellow | (231, 214, 60) |
| Water | Yellow | (235, 201, 70) |
| Hemoglobin | Purple | (209, 104, 208) |
| Myoglobin | Violet | (207, 71, 163) |
| SNCAGARLACG | Yellow | (240, 218, 68) |
| (SEQ ID NO 6) | | |
| NOTA- | Light | (239, 130, 158) |
| SNCAGARLACG | Purple | |

### Example 13

This example demonstrates the immobilization of the protein phosvitin to an alumina substrate to generate changes in the visually observed interference color, documented by R, G, B coordinates, when observed using white light through a polarizer at 750 from normal. Additionally, this example demonstrates the ability for phosvitin to act as an interface molecule for stable immobilization of a subsequent biomolecule to the aluminum oxide surface, namely platelet derived growth factor-BB (PDGF-BB), followed by its subsequent recognition by a specific antibody as demonstrated by a further colour shift.

Alumina substrates (120 nm of aluminum) were made and anodized as set forth in Example 1 and the protein was immobilized to the alumina surface to generate changes in the visually observed interference colours using white light was observed through a polarizer at 750 from normal. The protein was placed on the alumina surface, 18.5 μL of 200 mcg/ml in 150 mM NaCl, in a 100% relative humidity environment and was then rinsed thoroughly with deionized water. The phosvitin coated surface was then exposed to a glutaraldehyde solution, 5% mg/mL in HEPES buffer, pH 8.3. The surface was then rinsed with deionized water and a lysine cap was applied for 60 minutes (0.5 M lysine, pH 7.0) to terminate any remaining open glutaraldehyde active sites. Finally, anti-PDFG-BB IgG and a non-specific IgG were exposed to PDFG-BB coated areas. Results of various colour changes are provided in Table 13.

TABLE 13

| | Observed | RGB |
|---|---|---|
| Protein Surface | Colour | Coordinates |
| Blank slide | Orange | (240, 95, 180) |
| Water | Orange | (240, 91, 184) |
| Phosvitin (PSV) | Purple | (233, 83, 206) |
| PSV + PDGF-BB | Blue | (159, 136, 251) |
| PSV + PDGF-BB + Anti-PDGF-BB | Teal | (113, 196, 253) |
| IgG | | |
| PSV + PDGF-BB + non-specific | Blue | (161, 136, 252) |
| IgG | | |

### Example 14

This example demonstrates the use of nanoparticles of a reflective metal (gold) to visualize colour changes on the alumina thin film surface. Alumina substrates were made and anodized as set forth in Example 1. SARS CoV 2 nucleocapsid protein (NC) with a histidine tag was immobilized on a thin film alumina surface to generate changes in the visually observed interference color, documented by R, G, B coordinates, when observed using white light through a polarizer at 750 from normal. The protein was placed on the alumina surface, 18.5 μL of 25 mcg/ml in deionized water, in a 100% relative humidity environment and then rinsed thoroughly with deionized water. After immobilizing the protein on the surface, 40 nm gold nanoparticles (nanoComposix, San Diego USA) at 0.25 nanomolar concentration, 18.5 μL were added to the surface for 30 minutes and then rinsed with deionized water for 30 minutes. The colour change was then observed as was completed for the protein alone. The nanoparticles enhanced the eye visible change in the interference colour.

TABLE 14

| Protein Surface | Observed Colour | RGB Coordinates |
|---|---|---|
| Blank slide | Yellow | (231, 214, 60) |
| Water | Yellow | (235, 201, 70) |
| SARS CoV 2 NC | Tan | (234, 170, 86) |
| SARS CoV 2 NC + nanoparticle | Purple | (216, 132, 181) |

### INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention can be carried out using a large number of variations of the devices, device components, methods steps set forth in the present description. As will be obvious to one of skill in the art, methods and devices useful for the present methods can include a large number of optional composition and processing elements and steps.

Whenever a range is given in the specification, for example, a temperature range, a time range, a size range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A device for affinity binding, comprising:
an aluminum oxide surface; and
a biomolecule including a histidine rich domain, a metal binding domain or a metal binding moiety to immobilize the biomolecule on the aluminum oxide surface, wherein the histidine rich domain provides at least 4 consecutive histidine residues; and
wherein the metal binding domain or the metal binding moiety is selected from phosvitin, porphyrin-containing proteins and macromolecules, and nitrogen-containing macrocyclic aminopolycarboxylic acids.

2. The device of claim 1, wherein the biomolecule includes the histidine rich domain providing at least 4 consecutive histidine residues.

3. The device of claim 1, wherein the metal binding domain or the metal binding moiety is selected from phosvitin, porphyrin-containing proteins and macromolecules having interlinked pyrrole rings such as hemoglobin and myoglobin, cytochrome c, chlorophyll and verteprofin, and nitrogen-containing macrocyclic aminopolycarboxylic acids having a cyclic core providing at least 10 ring members of which at least 3 are nitrogen atoms such as NOTA, NOTP, DOTA, TRAP, and DATA.

4. The device of any one of claims 1-3, wherein the biomolecule is immobilized on the aluminum oxide surface.

5. The device of any one of claims 1-3, wherein the biomolecule and the aluminum oxide surface are provided as separate components for subsequent immobilizing.

6. The device of any one of claims 1-5, wherein the biomolecule is immobilized by physical adsorption.

7. A method of testing whether affinity binding has occurred to an analyte of interest, comprising:
a) providing an aluminum oxide surface;
b) providing a biomolecule specific for affinity binding to the analyte, the biomolecule including a histidine rich domain, a metal binding domain or a metal binding moiety to immobilize the biomolecule through physical adsorption on the aluminum oxide surface, wherein the histidine rich domain provides at least 4 consecutive histidine residues, and wherein the metal binding domain or the metal binding moiety is selected from phosvitin, porphyrin-containing proteins and macromolecules, and nitrogen-containing macrocyclic aminopolycarboxylic acids;
c) either i) immobilizing the biomolecule on the aluminum oxide surface and then contacting with a sample for affinity binding to the analyte, or ii) contacting the biomolecule with a sample for affinity binding to the analyte and then immobilizing the biomolecule and any affinity bound analyte on the aluminum oxide surface; and
d) detecting the presence of the analyte upon either i) binding of the analyte to the biomolecule immobilized on the aluminum oxide surface, or ii) immobilizing the biomolecule and any affinity bound analyte on the aluminum oxide surface.

8. The method of claim 7, wherein the biomolecule is a peptide tag specific to the analyte of interest, or specific to another biomolecule which in turn is specific to the analyte of interest.

9. The device or method of any one of claims 1-8, wherein:
the aluminum oxide surface is provided on a reflective metal capable of generating a colour when covered by a layer of aluminum oxide; and
the aluminum oxide surface is an anodized surface;
such that, when the aluminum oxide surface is contacted with the biomolecule, a colour change is detected denoting immobilizing of the biomolecule; and/or
such that, when an analyte for affinity binding to the biomolecule contacts the biomolecule immobilized on, or subsequently immobilized with the biomolecule on, the aluminum oxide surface, a colour change is detected denoting affinity binding of the biomolecule to the analyte.

10. The method of claim 9, wherein the biomolecule includes the histidine rich domain providing at least 4 consecutive histidine residues.

11. The method of claim 9, wherein the metal binding domain or the metal binding moiety is selected from phosvitin, porphyrin-containing proteins and macromolecules having interlinked pyrrole rings such as hemoglobin and myoglobin, cytochrome c, chlorophyll and verteprofin, and nitrogen-containing macrocyclic aminopolycarboxylic acids having a cyclic core providing at least 10 ring members of which at least 3 are nitrogen atoms such as NOTA, NOTP, DOTA, TRAP, and DATA.

12. The method of claim 10, wherein the histidine rich domain is located at one or more of a C-terminus of the biomolecule, an N-terminus of the biomolecule, or an accessible portion between the C-terminus and the N-terminus of the biomolecule.

13. The method of claim 12, wherein the histidine rich domain includes six or more consecutive histidine residues.

14. The method of claim 12, wherein the histidine rich domain includes a polyhistidine tag with a plurality of consecutive histidine amino acid residues at one or both of the C-terminus and the N-terminus of the biomolecule, wherein the plurality of consecutive histidine amino acid residues is 5-20, 6-10, 6-8 or 6.

15. The method of any one of claims 9-14, wherein the biomolecule and the analyte of interest are members of a binding pair selected from the group consisting of antibody-antigen, antibody-hapten, enzyme-substrate, enzyme-receptor, toxin-receptor, protein-protein, avidin-biotin, aptamer-aptamer target, and drug receptor-drug.

16. The method of any one of claims 9-15, wherein immobilizing is by physical adsorption in aqueous media.

17. The method of any one of claims 9-16, wherein the biomolecule is provided on the aluminum oxide surface as a continuous or discontinuous coating, as delineated spots or lines, or as an array.

18. The method of any one of claims 9-17, wherein the aluminum oxide surface is provided on a substrate is in the form of a particle, powder, thin film, slide, strip, filter, bead, magnetic bead, magnetic particle, or coating.

19. The method of claim 18, wherein the aluminum oxide surface is provided by sputtering, evaporating, casting or extruding aluminum metal or an aluminum alloy, which is further anodized to provide a porous anodized aluminum oxide surface.

20. The method of claim 18, wherein the aluminum oxide surface is provided by RF sputtering, reactive sputtering or chemical vapour depositing aluminum oxide onto the substrate.

21. The method of any one of claims 9-20, further comprising, after detecting, contacting with a further sample to test for a secondary analyte of interest, wherein the secondary analyte of interest is capable of binding to the analyte detected in step d).

22. The method of any one of claims 9-21, further comprising, after detecting, regenerating the aluminum oxide surface for reuse by:
i) removing the analyte and the histidine immobilized biomolecule by treating with an acid such as EDTA, H₂CO₃, or sodium bicarbonate; and
ii) optionally recharging the aluminum oxide surface with an oxidizing agent such as hydrogen peroxide.

23. The method of any one of claims 9-21, wherein detecting the presence of the analyte is enhanced by adding a nanoparticle of a reflective metal.

24. The device of any one of claims 1-9, in the form of a kit.

25. Use of a biomolecule including a histidine rich domain, a metal binding domain or a metal binding moiety to immobilize the biomolecule on an aluminum oxide surface, wherein the histidine rich domain provides at least 4 consecutive histidine residues; and
wherein the metal binding domain or the metal binding moiety is selected from phosvitin, porphyrin-containing proteins and macromolecules, and nitrogen-containing macrocyclic aminopolycarboxylic acids.

26. A method of testing whether affinity binding has occurred to an analyte of interest, comprising:
a) providing an aluminum oxide surface on a reflective metal capable of generating a colour when covered by a layer of aluminum oxide, the aluminum oxide surface being a porous anodized aluminum oxide surface having a refractive index in the range of 1.2 to 1.4 to provide a first eye visible interference colour;
b) providing a biomolecule specific for affinity binding to the analyte, the biomolecule including a binding domain or binding moiety to immobilize the biomolecule on the porous anodized aluminum oxide surface;
c) either i) immobilizing the biomolecule on the porous anodized aluminum oxide surface and then contacting with a sample for affinity binding to the analyte, or ii) contacting the biomolecule with a sample for affinity binding to the analyte and then immobilizing the biomolecule and any affinity bound analyte on the porous anodized aluminum oxide surface; and
d) detecting a subsequent eye visible interference colour different from the first eye visible interference colour denoting the presence of the analyte upon either i) binding of the analyte to the biomolecule immobilized on the porous anodized aluminum oxide surface, or ii) immobilizing the biomolecule and any affinity bound analyte on the porous anodized aluminum oxide surface.

27. The method of claim 26, wherein immobilizing the biomolecule on the porous anodized aluminum oxide surface produces a second eye visible interference colour different from the first eye visible interference colour denoting stable immobilizing of the biomolecule on the porous anodized aluminum oxide surface, and wherein the subsequent eye visible interference colour is different from the second eye visible interference colour denoting the presence of the analyte.

28. A method of testing whether affinity binding has occurred to an analyte of interest, comprising:
a) providing an aluminum oxide surface on a reflective metal capable of generating a colour when covered by a layer of aluminum oxide, the aluminum oxide surface being a porous anodized aluminum oxide surface;
b) providing a biomolecule specific for affinity binding to the analyte, the biomolecule including a binding domain or binding moiety to immobilize the biomolecule on the porous anodized aluminum oxide surface;
c) either i) immobilizing the biomolecule on the porous anodized aluminum oxide surface and then contacting with a sample for affinity binding to the analyte, or ii) contacting the biomolecule with a sample for affinity binding to the analyte and then immobilizing the biomolecule and any affinity bound analyte on the porous anodized aluminum oxide surface; and
d) detecting a subsequent eye visible interference colour different from the first eye visible interference colour denoting the presence of the analyte upon either i) binding of the analyte to the biomolecule immobilized on the porous anodized aluminum oxide surface, or ii) immobilizing the biomolecule and any affinity bound analyte on the porous anodized aluminum oxide surface;
wherein a combined thickness of the biomolecule and one or more layers of the affinity bound analyte is maintained in a range below 42 nm such that the subsequent eye visible interference colour is a first or second order interference colour.

29. The method of any one of claims 26-28, wherein the biomolecule includes a histidine rich domain providing at least 4 consecutive histidine residues to immobilize the biomolecule on the porous anodized aluminum oxide surface.

30. The method of any one of claims 26-28, wherein the biomolecule includes a carboxy rich domain to immobilize the biomolecule on the porous anodized aluminum oxide surface.

31. The method of any one of claims 26-28, wherein the biomolecule includes a metal binding domain or a metal binding moiety to immobilize the biomolecule on the porous anodized aluminum oxide surface.

32. The method of any one of claims 26-31, wherein detecting the presence of the analyte is enhanced by adding a nanoparticle of a reflective metal.


SEQUENCE LISTING (6 sequences)

SEQ ID NO: 1 - protein, 22 aa, synthetic construct
DYKDDDDKLALALALALALAHH

SEQ ID NO: 2 - protein, 23 aa, synthetic construct
DYKDDDDKLALALALALALAHHH

SEQ ID NO: 3 - protein, 24 aa, synthetic construct
DYKDDDDKLALALALALALAHHHH

SEQ ID NO: 4 - protein, 25 aa, synthetic construct
DYKDDDDKLALALALALALAHHHHH

SEQ ID NO: 5 - protein, 26 aa, synthetic construct
DYKDDDDKLALALALALALAHHHHHH

SEQ ID NO: 6 - protein, 11 aa, synthetic construct
SNCAGARLACG